(19) United States
(12) Patent Application Publication
(10) Pub. No.: US 2010/0105042 A1
(43) Pub. Date: Apr. 29, 2010

(54) NOVEL METHODS

(75) Inventors: Alexander H. Taylor, King of Prussia, PA (US); Delfi Krishna, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 12/520,244

(22) PCT Filed: Dec. 20, 2007
(86) PCT No.: PCT/US07/88310

§ 371 Date: Jun. 19, 2009

Related U.S. Application Data
(60) Provisional application No. 60/871,301, filed on Dec. 21, 2006.

(51) Int. Cl. C12Q 1/68

(52) U.S. Cl. 435/6

(57) ABSTRACT

The present invention provides methods for site-specifically integrating at least one first nucleic acid into a genome of at least one cell, comprising:
transforming said genome with a reporter nucleic acid construct that is linked to a first att site;
selecting at least one first transformed cell having at least one integrated reporter nucleic acid construct;
introducing said at least one first nucleic acid and a homologous recombination mediating enzyme into said cell wherein said at least one first nucleic acid comprises at least one second att site that is complementary to said first att site; and
maintaining said cell under conditions sufficient for said at least one first nucleic acid to integrate into said first att site producing at least one stably integrated cell.

### FIELD OF INVENTION

This present invention relates to methods of site-specifically integrating at least one nucleic acid into a genome of at least one cell.

### BACKGROUND OF THE INVENTION

Heterologous proteins are expressed in a variety of cell expression systems including bacterial, yeast and mammalian expression systems. For instance, monoclonal antibodies (IgG isotypes) are produced using a variety of expression systems including, host systems such as Chinese hamster ovary (CHO), NS0, hybridoma and myeloma cells or their derivatives. In many of these expression systems, nucleic acid sequences encoding all or part of the heterologous protein of interest are inserted into the genome of the host cell. The amount of expression of a heterologous protein of interest can often depend on where the nucleic acid encoding the protein is inserted in the genome of the host cell.

Such site-specific recombinase enzymes have long DNA recognition sites that are typically not present even in the large genomes of mammalian cells. However, it has been recently demonstrated that recombinase pseudo sites, i.e. sites with a significant degree of identity to the wild-type binding site for the recombinase, are present in these genomes (Thyagarajan, B., et al., Gene 244, 47-54 (2000)). Thus, it is possible to direct nucleic acids sequences encoding heterologous proteins to site specific regions of a mammalian genome.

The present invention discloses methods for site specifically directing nucleic acids encoding a heterologous product, for instance, a heterologous polynucleotide or polypeptide, into a genomic hot spot, thereby increasing heterologous product produced in the host cell.

### SUMMARY OF THE INVENTION

In one aspect of the present invention, methods are provided for site-specifically integrating at least one first nucleic acid into a genome of at least one cell, comprising:
transforming said genome with a reporter nucleic acid construct that is linked to a first att site;
selecting at least one first transformed cell having at least one integrated reporter nucleic acid construct;
introducing said at least one first nucleic acid and a homologous recombination mediating enzyme into said cell wherein said at least one first nucleic acid comprises at least one second att site that is complementary to said first att site; and
maintaining said cell under conditions sufficient for said at least one first nucleic acid to integrate into said first att site producing at least one stably intergrated cell.

### DETAILED DESCRIPTION OF THE INVENTION

### Glossary

As used herein “genomic hot spot” refers to a location in the genome of a cell at which transformed nucleic acid(s) expresses greater amounts of heterologous product for example, but not limited to, RNA or polypeptide when compared with the level of production of the same heterologous product when transformed in another location in the same genome. For instance, a nucleic acid encoding a heterologous polypeptide may be found to express larger amounts of heterologous polypeptide when transformed at one location in a host cell genome compared with its transformation at another location. Other aliases for a genomic hot spot include, but are not limited to, open chromatin and active chromatin. Open, active chromatin refers to chromatin that is in a de-condensed state and therefore accessible to the transcription factors that drive gene expression. Such transcriptionally active chromatin may also be referred to as euchromatin.

As used herein “att site” means site-specific recombination site. Bacteriophage integrate into a host chromosome by means of site-specific recombination between a locus on the phage chromosome known as the phage att site (attP) and a locus on the bacterial chromosome known as the bacterial att site (attB). The result of the recombination event is the formation of two hybrid att sites referred to as attL and attR.

As used herein “complementary att site” means two or more att sites capable of recombination with each other when in the presence of a homologous recombination mediating enzyme.

As used herein “pseudo-site” is a DNA sequence recognized by a homologous recombination mediating enzyme such that the recognition site differs in one or more base pairs from the wild-type enzyme recognition sequence and/or is present as an endogenous sequence in a genome and differs from the genome where the wild-type recognition sequence for the recombinase resides.

“Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site.

A recombination site “native” to the genome, as used herein, means a recombination site that occurs naturally in the genome of a cell (i.e., the sites are not introduced into the genome, for example, by recombinant means.)

As used herein “enhancer/promoter” means region(s) of DNA (1) that drive initiation of transcription by providing a binding site for RNA polymerase and associated specific essential transcription factors (the promoter), and/or (2) comprise regulatory DNA elements that interact with genes to enhance their expression (enhancer). Example promoters include, but are not limited to, the cmv immediate early promoter/enhancer, beta globin promoter, Rous sarcoma virus long terminal repeat promoter/enhancer, and the elongation factor alpha promoter/enhancer.

As used herein “mammalian cell” means mammalian cell lines capable of continuous anchorage dependent or suspension growth, and capable of supporting recombinant protein expression. Examples of mammalian cells, include, but are not limited to, chinese hamster ovary (CHO), PerC6, SP2/0, NS0, HeLa, Madin-Darby Canine Kidney, COS cells, baby hamster kidney cells, and NIH 3T3 cells.

As used herein “reporter nucleic acid construct” means a nucleic acid sequence that produces a selectable marker. Selectable markers include, but are not limited to, dihydrofolate reductase (dhfr), antibiotic resistance genes including, neomycin (neo), genticin, hygromycin B, puromycin, zeocin, and ampicillin, beta.-galactosidase, fluorescent protein, such as green florescent protein (GFP), secreted form of human placental alkaline phosphatase, beta-glucuronidase, yeast selectable markers leu 2-d and URA3, apoptosis resistant genes, and antisense oligonucleotides

“Host cell(s)” is a cell, including but not limited to a mammalian cell, insect cell, bacterial cell or cell of a microorganism, that has been introduced (e.g., transformed, infected or transfected) or is capable of introduction (e.g., transformation, infection or transfection) by an isolated and/or heterologous polynucleotide sequence.

“Transformed” or “transforming” as known in the art, is a modification of an organism's genome or episome via the introduction of isolated and/or heterologous DNA, RNA, or DNA-RNA hybrid, or to any other stable introduction of such DNA or RNA.

“Transfected” or “transfecting” as known in the art, is the introduction of isolated and/or heterologous DNA, RNA, or a DNA-RNA hybrid, into a host cell or microorganism, including but not limited to recombinant DNA or RNA

“Identity,” means, for polynucleotides and polypeptides, as the case may be, the comparison calculated using an algorithm provided in (1) and (2) below.

(1) Identity for polynucleotides is calculated by multiplying the total number of nucleotides in a given sequence by the integer defining the percent identity divided by 100 and then subtracting that product from said total number of nucleotides in said sequence, or:

nₙ≦xₙ−(xₙ·y),

wherein nₙ is the number of nucleotide alterations, xₙ is the total number of nucleotides in a given sequence, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding a polypeptide may create nonsense, missense or frameshift mutations in this coding sequence and thereby alter the polypeptide encoded by the polynucleotide following such alterations.

(2) Identity for polypeptides is calculated by multiplying the total number of amino acids by the integer defining the percent identity divided by 100 and then subtracting that product from said total number of amino acids, or:

nₐ≦xₐ−(xₐ·y),

wherein nₐ is the number of amino acid alterations, xₐ is the total number of amino acids in the sequence, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Heterologous(ly)” means (a) obtained from an organism through isolation and introduced into another organism, as, for example, via genetic manipulation or polynucleotide transfer, and/or (b) obtained from an organism through means other than those that exist in nature, and introduced into another organism, as for example, through cell fusion, induced mating, or transgenic manipulation. A heterologous material may, for example, be obtained from the same species or type, or a different species or type than that of the organism or cell into which it is introduced.

“Isolated” means altered “by the hand of man” from its natural state, has been changed or removed from its original environment, or both. For example, a polynucleotide or a polypeptide naturally present in a living organism is not “isolated,” but the same polynucleotide or polypeptide separated from the coexisting materials of its natural state is “isolated”, including but not limited to when such polynucleotide or polypeptide is introduced back into a cell, even if the cell is of the same species or type as that from which the polynucleotide or polypeptide was separated.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Polynucleotide(s)” generally refers to any polyribonucleotide or polydeoxyribonucleotide that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Recombinant expression system(s)” refers to expression systems or portions thereof or polynucleotides of the invention introduced (e.g, transfected, infected, or transformed) into a host cell or host cell lysate for the production of the polynucleotides and polypeptides of the invention.

As used herein “stably intergrated” or “stable intergration” means a transformed (or transformation of) a heterologous nucleic acid of interest into a host cell's chromosomal DNA such that long-term, reproducible expression is achieved.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

“Microorganism(s)” means a (1) prokaryote, including but not limited to, (a) Bacteria(I)(um), meaning a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (b) an archaeon, including but not limited to Archaebacter, and (2) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

As used herein “one type of heterologously expressed polypeptide” means all variants of a heterologously expressed polypeptide in a host cell, including all modified and unmodified heterologously expressed polypeptide.

As used herein “modified heterologously expressed polypeptide” means any heterologously expressed polypeptide or variant thereof wherein at least one amino acid of said polypeptide comprises a chemical modification. Chemical modifications may include, but are not limited to, methionine oxidation, glycosylation, gluconoylation, N-terminal glutamine cyclization and deamidation, and asparagine deamidation.

As used herein “recombinant antibody” means all variants of an antibody and/or fragment thereof expressed in a host cell from a recombinant expression system, including all modified and unmodified antibodies and/or fragment and/or variants thereof. Chemical modifications of a recombinant antibody may include, but are not limited to, methionine oxidation, glycosylation, gluconoylation, N-terminal glutamine cyclization and deamidation, and asparagine deamidation.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Antibodies typically comprise two heavy chains linked together by disulphide bonds and two light chains. Each light chain is linked to a respective heavy chain by disulphide bonds. Each heavy chain has at one end a variable domain followed by a number of constant domains. Each light chain has a variable domain at one end and a constant domain at its other end. The light chain variable domain is aligned with the variable domain of the heavy chain. The light chain constant domain is aligned with the first constant domain of the heavy chain. The constant domains in the light and heavy chains are not involved directly in binding the antibody to antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which typically include different antibodies directed against different epitopes, each monoclonal antibody is directed against a single epitope on the antigen.

“Antibody fragments” comprise a portion of a full length antibody, generally the antigen binding or variable domain thereof. Examples of antibody fragments include Fab, Fab′, F(ab′)₂, and Fv fragments; diabodies; linear antibodies; single-chain antibody molecules, and multispecific antibodies formed from antibody fragments.

The term “antigen binding fragment” refers to the domain of an antibody that specifically binds to an antigen.

The phrase “immunoglobulin single variable domain” refers to an antibody variable region (e.g., V_{H}, V_{HH}, V_{L}) that specifically binds an antigen or epitope independently of other V regions or domains; however, as the term is used herein, an immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). “Immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, as used herein refers to a mammalian immunoglobulin single variable domain polypeptide, which may be human, but also includes rodent (for example, as disclosed in WO 00/29004, the contents of which are incorporated herein by reference in their entirety) or camelid V_{HH} dAbs. Camelid dAbs are immunoglobulin single variable domain polypeptides which are derived from species including camel, llama, alpaca, dromedary, and guanaco, and comprise heavy chain antibodies naturally devoid of light chain: V_{HH}. V_{HH} molecules are about ten times smaller than IgG molecules, and as single polypeptides, they are very stable, resisting extreme pH and temperature conditions.

As used herein, “titer yield” refers to the concentration of a product (e.g., heterologously expressed polypeptide) in solution (e.g., culture broth or cell-lysis mixture or buffer) and may be expressed as mg/L or g/L. An increase in titer yield may refer to an absolute or relative increase in the concentration of a product produced under two defined set of conditions.

As used herein “harvesting” cells refers to collection of cells from cell culture. Cells may be concentrated during harvest to separate them from culture broth, for instance by centrifugation or filtration. Harvesting cells may further comprise the step of lysing the cells to obtain intracellular material, such as, but not limited to polypeptides and polynucleotides. It should be understood by the skilled artisan that certain cellular material, including but not limited to, heterologously expressed polypeptide, may by released from cells during culture. Thus, a product (e.g., a heterologously expressed polypeptide) of interest may remain in culture broth after cells are harvested.

In one aspect of the present invention, methods are provided for site-specifically integrating at least one first nucleic acid into a genome of at least one cell, comprising:
transforming said genome with a reporter nucleic acid construct that is linked to a first att site;
selecting at least one first transformed cell having at least one integrated reporter nucleic acid construct;
introducing said at least one first nucleic acid and a homologous recombination mediating enzyme into said cell wherein said at least one first nucleic acid comprises at least one second att site that is complementary to said first att site; and
maintaining said cell under conditions sufficient for said at least one first nucleic acid to integrate into said first att site producing at least one stably intergrated cell.

In another aspect, the first nucleic acid comprises a coding sequence, which may be present in a circular construct. The circular construct may be an expression cassette further comprises a bacterial origin of replication. The first nucleic acid may also comprise a control region, which is a promoter. The skilled artisan will understand various promoter/enhancer regions known in the art such as, but not limited to, the cmv immediate early promoter/enhancer, beta globin promoter, Rous sarcoma virus long terminal repeat promoter/enhancer, the elongation factor alpha promoter/enhancer, and the immunoglobulin promoters. The promoter may be operably linked to said coding sequence and wherein said coding sequence encodes a product. Examples of heterologously expressed products include polynucleotides and polypeptides. Polypeptides can comprise a whole or fragment of a heavy chain and/or a light chain of an immunoglobulin or a fragment thereof. Methods are also provided further comprising maintaining said at least one stably integrated cell under conditions sufficient to express said product. Said at least one cell may be a mammalian cell such as a Chinese hamster ovary cell or a genetically modified Chinese hamster ovary cell.

Also, provided are methods wherein the reporter nucleic acid construct encodes a selectable marker. Such a selectable marker provides for either positive or negative selection. Methods are also provided comprising expressing said selectable marker and comparing the amount of selectable marker produced by at least one first transformed cell of the selecting step with the amount of selectable marker produced by at least one second transformed cell of the selecting step wherein the first and second transformed cell produce the same selectable marker. As is understood in the art, selectable markers include, but are not limited to, dihydrofolate reductase (dhfr), antibiotic resistance genes including, neomycin (neo), geneticin, hygromycin B, puromycin, zeocin, and ampicillin, beta.-galactosidase, fluorescent protein, secreted form of human placental alkaline phosphatase, beta-glucuronidase, yeast selectable markers leu 2-d and URA3, apoptosis resistant genes, and antisense oligonucleotides. As is also understood in the art, cells can be sorted by a variety of means, including but not limited to, visual inspection or a cell sorter such as a BD FACS Aria, which can detect expression of a selectable marker.

Homologous recombination mediating enzyme of the present methods may be encoded on an expression cassette comprising a polynucleotide encoding said homologous recombination mediating enzyme, wherein said expression cassette is introduced into a host cell. Examples of a homologous recombination mediating enzyme include: λ bacteriophage integrase, φC31 phage recombinase, TP901-1 phage recombinase, R4 phage recombinase, meganuclease. Cre recombinase, Cre-like recombinase, Flp recombinase, and R recombinase.

Att sites are understood in the art to include, but are not limited to, wild-type attB, wild-type attP, pseudo-attb and pseudo-attP. Bacteriophage integrate into a host chromosome by means of a site-specific recombination, mediated by an integrase enzyme, between a locus on the phage chromosome known as the phage att site (attP) and a locus on the bacterial chromosome known as the bacterial att site (attB). The result of the recombination event is the formation of two hybrid att sites referred to as attL and attR. Pseudo att sites, found in the genome of some vertebrates, have sufficient homology to natural att sites such that integrase mediated recombination may occur. The first att site of the present invention may be, for example, an attP recombination site; while the second att site may be complementary to it and, therefore, an attB recombination site.

In another aspect, methods are provided wherein said reporter nucleic acid construct further comprises a third and a fourth att site, wherein said third and fourth att site are oriented with respect to said reporter nucleic acid construct such that said reporter nucleic acid construct is removed from said genome upon contacting said third and fourth att site with the recombinating meditating enzyme of the introducing step and wherein said first att site remains transformed in said genome. Said third att site may be an attP recombination site while said fourth att site may be an attB recombination site.

In another embodiment of the present invention, methods are provided for identifying a genomic hot spot in at least one cell comprising the steps of:
transforming the genome of a first cell and a second cell with a reporter nucleic acid construct that is linked to a first att site wherein said reported nucleic acid encodes a selectable marker;
expressing said selectable marker and comparing the amount of selectable marker produced by at least one first transformed cell with the amount of selectable marker produced by at least one second transformed cell wherein the first and second transformed cell produce the same selectable marker; and
selecting at least one first transformed cell having at least one integrated reporter nucleic acid construct by comparing the amount of selectable marker produced by said first and second transformed cell.

In one aspect, the selectable marker provides for either positive or negative selection. In another aspect, the reporter nucleic acid construct is transformed into said first cell and said second cell in the presence of a homologous recombination mediating enzyme. Homologous recombination mediating enzyme can be selected from the group of: λ bacteriophage integrase, φC31 phage recombinase, TP901-1 phage recombinase, R4 phage recombinase, meganuclease. Cre recombinase, Cre-like recombinase, Flp recombinase, and R recombinase.

The first att site may be an attP recombination site. In addition, the reporter nucleic acid may be further linked to a second att site. The second att site may be an attB recombination site. Transformed cells may be mammalian cells, which includes, but is not limited to, Chinese hamster ovary cells.

In yet another aspect, the reporter nucleic acid construct further comprises a third and a fourth att site, wherein said third and fourth att site are oriented with respect to said reporter nucleic acid construct such that said reporter nucleic acid construct is removed from said genome upon contacting said third and fourth att site in the presence of homologous recombination meditating enzyme and wherein said first att site remains transformed in the genome of said at least one cell. The third att site may be an attP recombination site and the fourth att site may be an attB recombination site. The report nucleic acid may encode green florescent protein.

The following examples illustrate various aspects of this invention. These examples do not limit the scope of this invention which is defined by the appended claims

### EXAMPLES

### Example 1

### Design and Preparation of a Reporter Plasmid DNA Vectors

A reporter plasmid is assembled with the following genetic elements:

(a) A pUC 19-based plasmid backbone with all of the genetic elements required for plasmid replication in bacterial hosts, such as an origin of replication and ampicillin resistance selectable marker; a polylinker is also included to enable subsequent subcloning steps.

(b) A destabilized green fluorescent protein (GFP) coding sequence is inserted between the CMV promoter and a bovine growth hormone poly A addition signal sequence, thereby allowing expression in a mammalian host.

(c) A bacteriophage φc31 integrase attB site.

(d) A neomycin resistance (NEO) coding sequence is inserted between a beta globin promoter and a bovine growth hormone poly A addition signal sequence, thereby allowing expression in a mammalian host and is used as a selectable marker for stable transfectants.

### Example 2

### Construction of an attB-Marked Transfectoma Library

A transfectoma library is created in CHO DG44 cells by transfecting 10 μg of the reporter plasmid DNA, into the CHO cells. Plasmid DNA is linearized by restriction endonuclease treatment and the cleaved product is ethanol precipitated and then resuspended in a 10 μl volume of water. The CHO cells would then be transfected with the linearized DNA and cationic lipid transfer reagent.

After transfection, the CHO cells are seeded at a cell density of 0.5×10⁶ cells/ml and maintained at 37° C. in static culture using normal growth medium for 48 hours. 400 μg/ml Geneticin® is then added to the medium to mediate selection of stably transfected CHO cells. The cultures are maintained under these conditions for three weeks or until neomycin resistant colonies begin to appear and then maintained by twice weekly passaging seeding at 0.5×10⁶ cells/mL.

### Example 3

### Fluorescence Activated Cell Sorting (FACS)

FACS is used to isolate the top 0.1% highest GFP expressers. The stable CHO transfectoma library of cells are first examined for GFP fluorescence to establish the appropriate gating parameters and sorting logic gates using an analytical flow cytometer.

20×10⁶ cells are suspended in 1 ml fresh media and stained with propidium iodide for live/dead cell exclusion. The entire cell population are gated to exclude dead cells and cell doublets. Fluorescence of live singlet cells is examined and gates are created to sort 0.1% of the brightest cells into a separate tube. The sorted cells are expanded in growth medium until enough cells accumulate, and a second round of FACS is performed essentially as before, but this time the top 0.1% brightest cells are sorted into individual 96-wells containing a feeder layer and medium supplemented with conditioned medium. Clonal colonies arise after 3-4 weeks of culture and are expanded and analyzed for GFP expression using flow cytometery. The clones exhibiting the greatest level of GFP expression are selected for further analysis.

### Example 4

### Design and Preparation of Variants of Bacteriophage φc31 Integrase

The wild type bacteriophage φc31 integrase coding region is synthesized from sequences deposited in genbank by Geneart, Inc., cloned into a shuttle vector and sequenced. Three versions of bacteriophage φc31 integrase are assembled as follows:
(a) wild type bacteriophage φc31 integrase DNA;
(b) codon optimized bacteriophage φc31 integrase DNA encoding wild type bacteriophage φc31 integrase protein; and
(c) codon optimized bacteriophage φc31 integrase DNA encoding wild type bacteriophage φc31 integrase protein, with a nuclear localization signal peptide appended to the c-terminus of the protein.
   The synthetic gene sequences are cloned into a mammalian expression vector with the following genetic elements:
(a) pUC 19-based plasmid backbone with all of the genetic elements required for plasmid replication in bacterial hosts, such as an origin of replication and ampicillin resistance selectable marker; a polylinker is also included to enable subsequent subcloning steps; and
(b) multiple cloning site 5′ flanked with a CMV promoter and 3′ flanked with a bovine growth hormone poly A addition signal sequence, thereby allowing transient expression in a mammalian host.

### Example 5

### Design and Preparation of a Directed Integration Vector

A directed integration plasmid is assembled with the following genetic elements:
(a) pUC 19-based plasmid backbone with all of the genetic elements required for plasmid replication in bacterial hosts, such as an origin of replication and ampicillin resistance selectable marker; a polylinker is also included to enable subsequent subcloning steps;
(b) expression cassette comprising an antibody heavy- and light-chain coding region, each flanked 5′ by a CMV promoter and 3′ by a bovine polyA recognition sequence;
(c) bacteriophage φc31 integrase attP site; and
(d) expression cassette comprising a dihydrofolate reductase coding region, flanked 5′ by a β-globin promoter and 3′ by a bovine polyA recognition sequence.

### Example 6

### Co-Transfection of attB-Marked, High GFP Expressing CHO Clones (See Example 3) with the Bacteriophage φc31 Integrase Expression Vector (See Example 4) and the Directed Integration Vector (See Example 5)

The product of example 5 provides clones which are stably transfected with GFP at genetic loci that support high levels of gene expression, and would also be marked by genetic linkage with a bacteriophage φc31 integrase attB site. Accordingly, targeted integration of the targeting vector could subsequently be carried out by co-transfection of such cells with the bacteriophage φc31 integrase expression vector (which transiently provides for expression of the bacteriophage φc31 integrase enzyme) and the directed integration vector (which supplies an attP site which is complemetary to the chromosomal attB site previously introduced). The targeted integration event is mediated by the transiently expressed bacteriophage φc31 integrase by the molecular recognition of the respective attB and attP sites, precise DNA strand cutting, DNA cross-over strand exchange, and ligation of the recombination products.

The co-transfection is carried out by first linearizing 10 μg each of the two plasmids, ethanol precipitation and resuspending each in 10 μl of water, and finally simultaneous cationic lipid transfer transfection. After transfection, the CHO cells are seeded at a cell density of 0.5×10⁶ cells/ml and maintained at 37° C. in static culture using normal growth medium for 48 hours. Stable integration of the targeting vector is then selected for by replacing the normal growth medium with a growth medium deficient in nucleosides, which selects for the presence of the DHFR gene. The cultures are maintained under these conditions for three weeks or until DHFR positive colonies begin to appear. The emerging cultures are then maintained indefinitely by passaging twice weekly, seeding at 0.5×10⁶ cells/mL.

### Example 7

### Screening of Directed Integration Library

The DHFR positive site directed integration library obtained in example 6 is expected to contain a majority of clones that have undergone random integration of the targeting vector, and most likely result in low expression of the MAb genes, as well as targeted insertion events which result in high level expression of the MAb genes. Accordingly, to identify the desired clones expressing high levels of the MAb gene, a screening step is employed. The DHFR positive site directed integration library is treated with a fluoresceinated antibody specific for the MAb gene product, washed and analysed on the FACS. Cells that stain brightly for surface MAb express the MAb at proportionately high levels and are sorted into individual 96-wells containing a feeder layer and medium supplemented with conditioned medium. Clonal colonies would arise after 3-4 weeks of culture and are expanded and analysed for MAb expression using ELISA. The clones exhibiting the greatest level of MAb expression are selected for further analysis.

### Example 8

### Amplification of Integrated DNA

The clones obtained from example 7 harbor only a single copy of the MAb gene. Due to the genetic linkage of the DHFR and the MAb heavy and light chain genes, further enhancement of the MAb gene expression may be obtained by genetic amplification. Selected clones are seeded into 96-well cultures in the presence of 5 nM methotrexate (MTX). Only those CHO cells that undergo spontaneous amplification of the DHFR gene are resistant to the MTX and could proliferate, forming colonies. The resulting MTX resistant colonies would then be screened for MAb productivity as before. The MTX amplification step can be iteratively applied, in each instance using increasing concentration of MTX, leading to even further MAb expression levels.

### Example 9

### CHO cell Transfection and Selection for Green Florescent Protein (GFP) Expression

CHO-DG44 cells were transfected with a plasmid comprising a gene encoding destabalized GFP protein (dsGFP, approximate half life 1-2 hr) and neomycin in the presence or absence of a plasmid comprising a gene encoding an optimized version of integrase φC31. Forty-eight hours post transfection, the cells were exposed to selective media containing geneticin to select for stable transfectants. The cells were incubated in geneticin containing media for approximately 3 weeks following which the bulk population of stable transfectants were analyzed for GFP expression using flow cytometry.

In order to analyze the populations, gates at various intervals of GFP expression were constructed. Transfection in the presence of integrase φC31 yielded 34-78% more GFP positive events at each GFP interval (Table 1) indicating that the integrase φC31 is functional in this system.

TABLE 1

| | EVENTS | EVENTS | |
|---|---|---|---|
| GATES | NO INTEGRASE | INTEGRASE | % OF EVENTS HIGHER |
| P2 | 291 | 473 | 39% |
| P3 | 190 | 287 | 34% |
| P4 | 129 | 250 | 49% |
| P5 | 111 | 194 | 43% |
| P6 | 70 | 124 | 44% |
| P7 | 28 | 74 | 62% |
| P8 | 6 | 27 | 78% |

The bulk population of stable transfectants obtained in the presence or absence of integrase φC31 were sorted using the BD FACS Aria into two separate populations. The sorted populations were defined by interval gates based on GFP expression as P2 (bright) and P3 (dim), respectively.

The sorted populations were grown and expanded into shake flasks followed by analysis for GFP expression using flow cytometry. Susequently, BD FACS was used to isolate single cell clones with high GFP expression. The sort criteria for cells expressing high levels of GFP was defined using interval gate P2. Single cell clones generated from the above process were allowed to expand into shake flasks. The clones were then analyzed for GFP expression. Table 2 depicts the raw data (mean GFP expression of each clone) obtained for individual clones that were generated via transfection in the presence or absence of integrase. The quality of clones obtained in the presence of φC31 (average mean GFP expression 26667) is far superior as compared to clones obtained by random integration of GFP into the CHO cell genome (average mean GFP expression 752). Clones were defined to be “positive” if greater than 90% of the events in the clonal population were above background fluorescence. Based on that criterion, 3 out of 24 (12%) random GFP integrants are considered positive. In contrast, for clones generated in the presence of integrase φC31, 12 out of 23 (52%) were “positive.”

TABLE 2

| CLONE | NO INTEGRASE | INTEGRASE |
|---|---|---|
| 1 | 2926 | 43,561 |
| 2 | 652 | 60,993 |
| 3 | 331 | 50,000 |
| 4 | 646 | 29,336 |
| 5 | 362 | 36,304 |
| 6 | 360 | 40,546 |
| 7 | 111 | 86,328 |
| 8 | 2366 | 17,437 |
| 9 | 521 | 2434 |
| 10 | 516 | 3446 |
| 11 | 60 | 45,520 |
| 12 | 139 | 35,890 |
| 13 | 340 | 108,758 |
| 14 | 507 | 513 |
| 15 | 229 | 1925 |
| 16 | 816 | 3992 |
| 17 | 284 | 2717 |
| 18 | 3994 | 557 |
| 19 | 87 | 855 |
| 20 | 190 | 5356 |
| 21 | 742 | 2385 |
| 22 | 282 | 30,300 |
| 23 | 1173 | 4192 |
| 24 | 425 | n/a |

The quality of clones obtained in the presence of integrase φC31 strongly suggests that the integrase is capable of directing the GFP plasmid to integrate and express from transcriptional sites that are highly active within the CHO cell genome. In contrast, random integration of the GFP cassette into the genome results in high GFP expression less frequently.

1.-25. (canceled)

26. A method of identifying a genomic hot spot in at least one cell comprising the steps of:
transforming the genome of a first cell and a second cell with a reporter nucleic acid construct that is linked to a first aft site wherein said reported nucleic acid encodes a selectable marker;
expressing said selectable marker and comparing the amount of selectable marker produced by at least one first transformed cell with the amount of selectable marker produced by at least one second transformed cell wherein the first and second transformed cell produce the same selectable marker; and
selecting at least one first transformed cell having at least one integrated reporter nucleic acid construct by comparing the amount of selectable marker produced by said first and second transformed cell.

27. The method of claim 26, wherein the selectable marker provides for either positive or negative selection.

28. The method of claim 26, wherein said reporter nucleic acid construct is transformed into said first cell and said second cell in the presence of a homologous recombination mediating enzyme.

29. The method of claim 28 wherein said homologous recombination mediating enzyme is selected from the group of: λ bacteriophage integrase, φC31 phage recombinase, TP901-1 phage recombinase, R4 phage recombinase, meganuclease. Cre recombinase, Cre-like recombinase, Flp recombinase, and R recombinase.

30. The method of claim 26 wherein said first aft site is an attP recombination site.

31. The method of claim 26, wherein said reporter nucleic acid is further linked to a second aft site.

32. The method of claim 31 wherein said second aft site is an attB recombination site.

33. The method of claim 28, wherein said at least one cell is a mammalian cell.

34. The method of claim 32, wherein said at least one cell is a Chinese hamster ovary cell.

35. The method of claim 31, wherein said reporter nucleic acid construct further comprises a third and a fourth aft site, wherein said third and fourth aft site are oriented with respect to said reporter nucleic acid construct such that said reporter nucleic acid construct is removed from said genome upon contacting said third and fourth aft site in the presence of homologous recombination meditating enzyme and wherein said first aft site remains transformed in the genome of said at least one cell.

36. The method of claim 35 wherein said third aft site is an attP recombination site.

37. The method of claim 35 wherein said fourth aft site is an attB recombination site.

38. The method of claim 26, wherein the report nucleic acid encodes green florescent protein.
